(19) United States
(12) Patent Application Publication
(10) Pub. No.: US 2025/0064677 A1
(43) Pub. Date: Feb. 27, 2025

(54) SINGLE-USE FLUID CONTAINER WITH RETURN PORTS

(71) Applicant: AQUASYN, LLC, Carson City, NV (US)

(72) Inventor: Michael C. Gagne, Reno, NV (US)

(73) Assignee: AQUASYN, LLC, Carson City,, NV (US)

(21) Appl. No.: 18/943,659

(22) Filed: Nov. 11, 2024

Related U.S. Application Data
(63) Continuation of application No. PCT/US2023/066800, filed on May 9, 2023.
(60) Provisional application No. 63/340,756, filed on May 11, 2022.

(51) Int. Cl. A61J 1/10; A61J 1/14; A61J 1/20

(52) U.S. Cl. CPC A61J 1/10; A61J 1/1475; A61J 1/2062

(57) ABSTRACT

A single-use fluid container includes a flexible bag having a bottom surface and one or more side surfaces. A port or aperture is formed in the bottom surface of the flexible bag that serves as an inlet port to a multi-outlet pump that is fluidically coupled to the flexible bag. The flexible bag includes one or more return ports disposed in one or more side surfaces and/or bottom surface of the flexible bag, the one or more return ports having a valve therein. One or more of the outlets of the pump are connected to conduit or tubing that also connect to one or more of the return ports. Fluid contained in the flexible bag can then be recirculated back into the flexible bag for mixing and/or agitation. Optional sparger ports may be provided for the addition of a sparging gas.

### RELATED APPLICATIONS

This Application is a continuation of International Application No. PCT/US2023/066800 filed on May 9, 2023 which itself claims priority to U.S. Provisional Patent Application No. 63/340,756 filed on May 11, 2022, which are hereby incorporated by reference. Priority is claimed pursuant to 35 U.S.C. § 120, 119 and any other applicable statute.

### TECHNICAL FIELD

The technical field generally relates to containers used in connection with biopharmaceutical, pharmaceutical, and biotechnology applications. More specifically, the technical field relates to flexible containers such as bags that hold fluids therein and include a plurality of ports formed therein that are used as fluid return flow paths to recirculate and/or mix fluid as well as optional sparger ports.

### BACKGROUND

Biopharmaceutical, pharmaceutical, and biotechnology applications have historically used reusable devices in the research and manufacturing processes associated with these industries. These reusable devices, however, must be cleaned and sterilized between uses. This was typically accomplished using a sterilization agent (e.g., steam) that was introduced into the reusable devices. The sterilization process, however, is time-consuming and introduces considerable cost into the process. Moreover, there is the possibility that sterilization was not effective, leading to contamination issues. More recently, these industries are moving toward using single-use components instead of reusable devices for parts and components that come into contact with reagents and products. An advantage of single-use or disposable products is that it eliminates the need for cleaning and sterilizing devices in between uses.

Containers for holding fluid are regularly used in the biopharmaceutical, pharmaceutical, and biotechnology fields. These containers may be rigid or flexible depending on the particular process or application. Sometimes, fluid within the containers needs to be mixed or agitated. One common way to aid in mixing is to add a mixing unit that includes a shaft and impeller that extends downward into the interior of the container and rotates to mix fluid. Flexible fluid containers such as bags are also used in biopharmaceutical, pharmaceutical, and biotechnology applications. These flexible fluid containers may be single-use products which can be discarded after use. Fluid containers such as bags can be coupled directly or indirectly to pumps to evacuate fluid from the interior of the bag and pump this fluid to other processes. There is a need for solutions to incorporate mixing and/or agitation and sparging functionality into flexible fluid containers such as bags.

### SUMMARY

In one embodiment, a single-use flexible fluid container or bag is disclosed that includes one or more ports that are used to fill the flexible fluid container or bag with fluid. One or more of these ports may also act to vent air/gases when fluid is filling the flexible fluid container or bag. The ports may also vent gas that is introduced into the flexible container or bag or generated natively therein. The flexible fluid container or bag further includes a bottom surface having a port or aperture formed therein. The port or aperture is fluidically coupled to a multi-outlet pump. The flexible fluid container contains one or more return ports formed therein for receiving fluid from the multi-outlet pump. The multi-outlet pump includes conduits or tubing that connect at least some of the outlets of the multi-outlet pump to the return port(s). The return port(s) may include respective valves incorporated therein to control the flow of fluid into the flexible fluid container or bag with fluid. The valves may include, for example, bleed valves, sample valves, ball valves, plug valves, check-valves, butterfly valves, pinch valves, or fluid diode-based valves. The return port(s), in one embodiment, are located close to or at the bottom of the flexible fluid container or bag. This includes locating the return port(s) in the sides near the bottom and/or in the bottom surface of the flexible fluid container or bag. The multiple return ports may be located at the same or different elevations within the flexible fluid container or bag. Additional inlet ports may be provided to fill the flexible fluid container.

The return ports integrated into the flexible fluid container or bag may include a connector or end that allows conduit or tubing to be easily connected. For example, the return ports may include a barbed end that interfaces with an end of the conduit or tubing. In another embodiment, the ports described herein may include a sanitary clamp port fitting or end.

In some embodiments, the return ports may be configured to direct the returning flow of fluid into the interior of the flexible fluid container or bag in a directional manner. For example, the orientation of the return ports may be oriented to create a mixing or turbulent fluid environment within the interior of the flexible fluid container or bag. In some embodiments, the valves are used to prevent fluid contained in the flexible fluid container or bag from exiting the flexible fluid container or bag. For example, after fluid has been recirculated to the flexible fluid container or bag via the ports, the valves of the return ports may be closed (either by manual operation or through the valve design (e.g., check-valve, diode valve, etc.). The conduit or tubing can then be removed from the flexible fluid container or bag.

In some embodiments, one or more sparger ports are located in the flexible fluid container or bag that allow the introduction of sparging gas into the flexible fluid container or bag. The sparger ports may be located in the bottom and/or side surfaces of the flexible container or bag. In some embodiments, the sparger ports may include a sparger element that is inserted into a port (e.g., tri-clamp port) that is disposed on the flexible container or bag. The sparger element can be fixed to the port using, for example, a sanitary clamp. Different sparger elements may be swapped into or out of the sparger port.

In one embodiment, a single-use flexible fluid container is disclosed that includes a flexible bag having a bottom surface and one or more side surfaces. A port or aperture is formed in the bottom surface of the flexible bag and a plurality of return ports are disposed in one or more side surfaces and/or a bottom surface of the flexible bag, each return port comprising a valve therein. The single-use flexible fluid container may be removably secured to a pump (e.g., a pump head) that is used to pump or recirculate fluid originating from the flexible bag back into the flexible bag via one or more of the return ports. Conduit or tubing connects the pump outlets to the respective return ports.

In another embodiment, a system for mixing fluids includes a flexible bag having a bottom surface and one or more side surfaces, wherein the bottom surface comprises a port or aperture formed therein. One or more return ports are disposed in one or more side surfaces and/or a bottom surface of the flexible bag, the one or more return ports having a valve therein. The system includes a pump having an inlet and a plurality of outlets, wherein the inlet of the pump is in fluid communication with the port or aperture formed in the bottom surface and wherein one or more of the plurality of outlets are connected to respective conduits or tubing that are further fluidically connected to the one or more return ports.

In another embodiment, a method of using a single-use flexible fluid container includes: providing a flexible bag having a bottom surface and one or more side surfaces and a port or aperture formed in the bottom surface of the flexible bag, wherein a plurality of return ports are disposed in one or more side surfaces or a bottom surface of the flexible bag, each return port comprising a valve therein; securing a pump to the flexible bag, wherein the pump comprises an inlet and a plurality of outlets, wherein the inlet is in fluid communication with the port or aperture formed in the bottom surface of the flexible bag; securing tubing or conduits to one or more of the plurality of outlets and to one or more of the plurality of return ports; and operating the pump to recirculate fluid into the flexible bag. Optionally, gas may be introduced into the flexible bag using one or more sparger ports.

### BRIEF DESCRIPTION OF THE DRAWINGS

FIG. 1A is a front view of a single-use flexible container according to one embodiment.

FIG. 1B is a side view of the single-use flexible container of FIG. 1A.

FIG. 1C is a back view of the single-use flexible container of FIG. 1A.

FIG. 1D illustrates an enlarged view of detail 1D of FIG. 1B.

FIG. 1E illustrates a bottom view of the single-use flexible container of FIG. 1A.

FIG. 2A illustrates a front view of a single-use flexible container according to another embodiment.

FIG. 2B is a side view of the single-use flexible container of FIG. 2A.

FIG. 2C is a back view of the single-use flexible container of FIG. 2A.

FIG. 3A illustrates a front view of a single-use flexible container according to another embodiment. The single-use flexible container is secured to a pump.

FIG. 3B is a side view of the single-use flexible container of FIG. 3A. Conduit or tubes connect the outlets of the pump to the return ports of the flexible bag.

FIG. 3C is a back view of the single-use flexible container of FIG. 3A.

FIG. 4A illustrates a front view of a single-use flexible container according to another embodiment. In this embodiment, return ports are located in the bottom surface of the flexible bag.

FIG. 4B is a side view of the single-use flexible container of FIG. 4A.

FIG. 4C is another front view of the single-use flexible container of FIG. 4A.

FIG. 5A is a perspective view of one embodiment of a return port that includes a valve therein (check-valve).

FIG. 5B is a cross-sectional view of the return port and valve of FIG. 5A.

FIG. 6A is a perspective view of one embodiment of a return port that includes a valve therein (sample valve).

FIG. 6B is a cross-sectional view of the return port and valve of FIG. 6A.

FIG. 7A is a perspective view of one embodiment of a return port that includes a valve therein (ball valve).

FIG. 7B is a cross-sectional view of the return port and valve of FIG. 7A.

FIG. 8A is a perspective view of one embodiment of a return port that includes a valve therein (plug valve).

FIG. 8B is a cross-sectional view of the return port and valve of FIG. 8A.

FIG. 9A illustrates a front view of a single-use flexible container according to another embodiment. In this embodiment, sparger ports are illustrated in the bottom and side surfaces of the flexible bag for introducing gas.

FIG. 9B is a cross-sectional view of the flexible container of FIG. 9A taken along the line 9B-9B of FIG. 9A.

FIG. 9C is a detailed view of detail 9C of FIG. 9A showing the sparger port.

FIG. 10A illustrates a front view of a single-use flexible container according to another embodiment. In this embodiment, sparger ports are illustrated in the bottom and side surfaces of the flexible bag for introducing gas. The sparger ports include a sparger element that is clamped to a tri-clamp port located on the flexible bag.

FIG. 10B is a cross-sectional view of the flexible container of FIG. 10A taken along the line 10B-10B of FIG. 10A.

FIG. 10C is a detailed view of detail 10C of FIG. 10B showing the sparger port.

FIG. 10D is a perspective view of the sparger element.

FIG. 11A illustrates a front view of a single-use flexible container according to another embodiment. In this embodiment, a valve is interposed between the pump head of a pump and a port disposed on the bottom of the flexible bag.

FIG. 11B is a cross-sectional view of the flexible container of FIG. 11A taken along the line 11B-11B of FIG. 11A.

### DETAILED DESCRIPTION OF THE ILLUSTRATED EMBODIMENTS

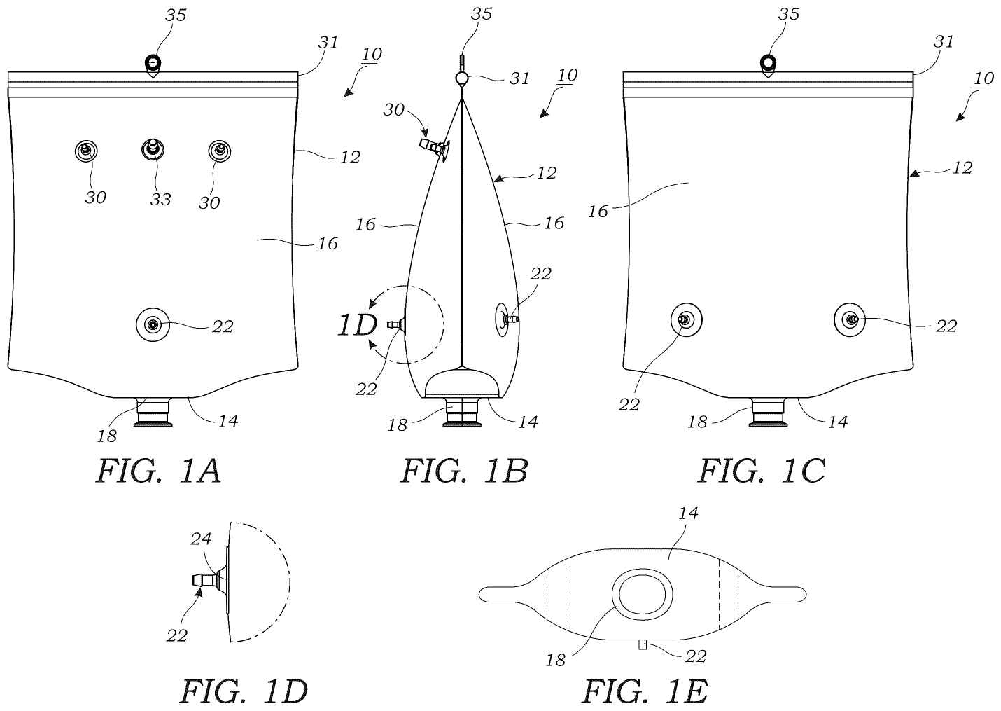

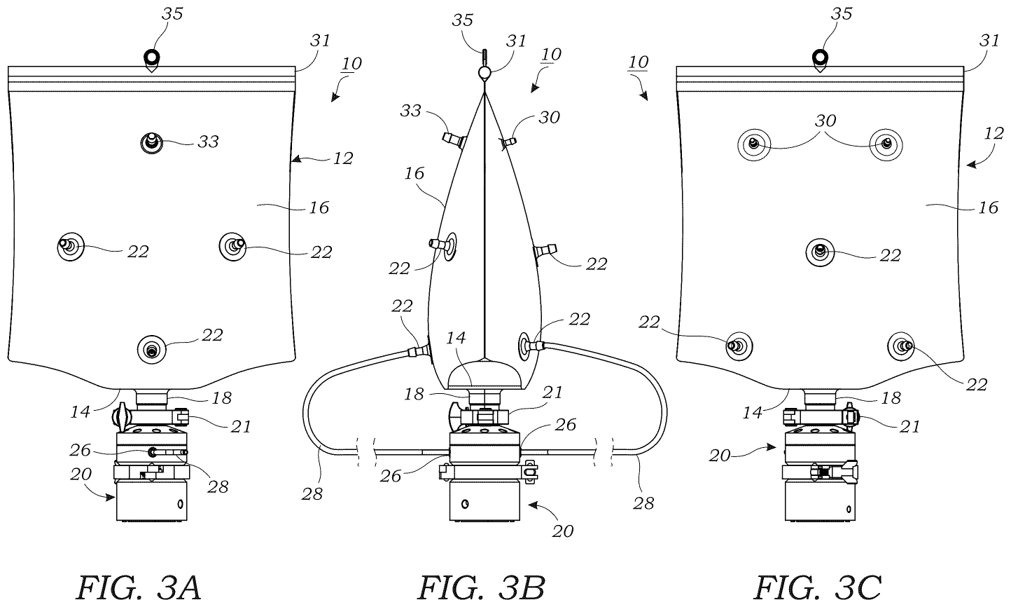

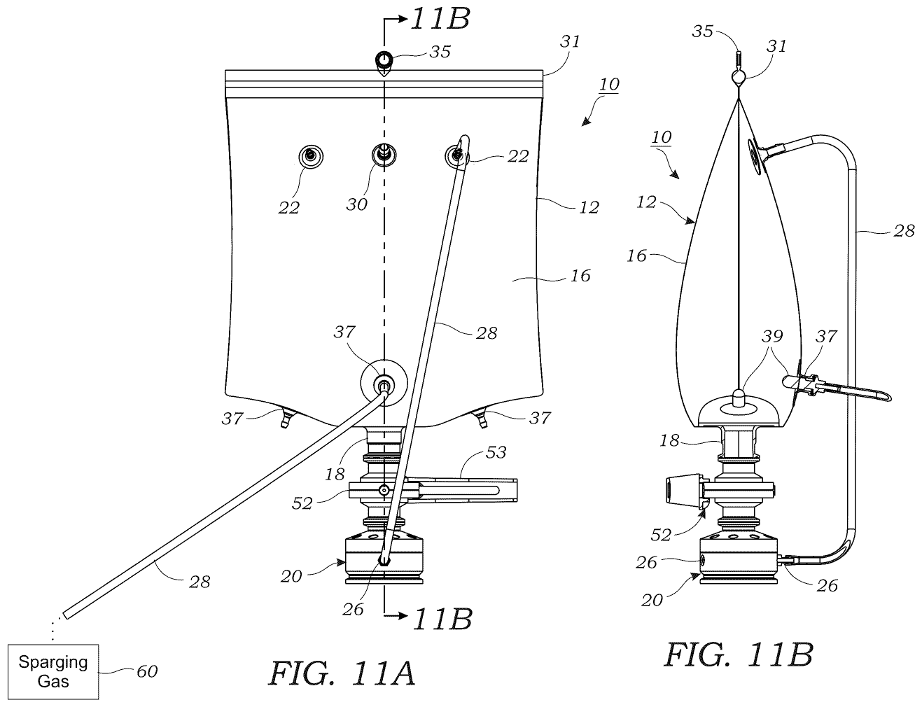

FIGS. 1A-1E illustrates one embodiment of the single-use flexible container 10. The flexible container 10 in this embodiment includes a flexible bag 12 having a bottom surface 14 and one or more side surfaces 16. In some embodiments, the flexible bag 12 may also have a top surface (not shown). A port or aperture 18 is formed in the bottom surface 14 of the flexible bag 12 that serves as an inlet port to a multi-outlet pump 20 (FIGS. 3A-3C, 11A, 11B). The port or aperture 18 can be integrally formed with the flexible bag 12 or a separate port 18 may be secured to the bottom surface 14 and an aperture formed therein provides fluid access to the port 18. The port or aperture 18 may include a variety of sizes (e.g., 1.5 inches, 3 inches, 4 inches, and the like). The port 18 may include a flanged port or a port with a sanitary connector that is configured to be secured to the multi-outlet pump 20 using mating flanged surface on the multi-outlet pump 20. The two components (pump 20 and flexible bag 12) may be secured to one another via a sanitary clamp 21 or the like as seen in FIGS. 3A-3C. In other embodiments, the pump 20 may be directly integrated with or bonded to the flexible bag 12. For example, a pump head of the pump 20 may be integrated or bonded to the bottom surface 14 of the flexible bag 12 and the port or aperture 18 provides fluid access to the inlet of the pump head of the pump 20. The pump head of the pump 20 may also be bonded or integrated with a flanged port 18 that is located in the bottom surface 14 of the flexible bag 12. Exemplary pumps 20 that may be used with the single-use flexible containers 10 disclosed herein include those disclosed in, for example, U.S. Pat. No. 11,331,248 and International Patent Application No. PCT/US2021/015917, published as WO 2021/158448, which are incorporated by reference herein.

The flexible bag 12 includes one or more of return ports 22 disposed in one or more side surfaces of the flexible bag 12 and/or the bottom surface 14 of the flexible bag 12. Some or all of the return ports 22 have a valve 24 therein (best seen in FIGS. 1D, FIGS. 5A, 5B, 6A, 6B, 7A, 7B, 8A, 8B). The return ports 22 allow for fluid to be recirculated or otherwise fed to the flexible bag 12. With reference to FIGS. 3A and 3B, one or more of the outlets 26 of the pump 20 are connected to conduit or tubing 28 that also connect to one or more of the return ports 22. Fluid contained in the flexible bag 12 can then be recirculated using the multi-outlet pump 20 back into the flexible bag 12 for mixing and/or agitation. In one particular embodiment, a single segment of conduit or tubing 28 connects at one end to the outlet 26 of the pump 20 (or a connector coupled to the outlet 26) and at the other end to one of the return ports 22. For example, in one implementation, each outlet 26 returns fluid to a different return port 22. However, not all outlets 26 necessarily need to return back to the flexible bag 12. Some outlets 26 may be directed to other processes or devices. The contents of the flexible bag 12, for example, may be evacuated via one of the outlets 26 of the pump 20. In addition, the conduit or tubing 28 may direct fluid to another device or processing unit (e.g., a filter) prior to returning to the return port 22. In this regard, the conduit or tubing 28 may be interrupted by another process or device and multiple conduits or tubes 28 are used in the return path.

The flexible bag 12 may further include one or more inlet ports 30 that are used to input fluid into the flexible bag 12. These inlet ports 30 may also include a valve 24 therein to allow fluid into the flexible bag 12 but not let fluid leave. The flexible bag 12 may also include one or more vent ports 33. The vent ports 33 are similar to the return ports 12 and/or the inlet ports 30 but do not include a valve therein. The vent ports 33 may include a filter material that prevents contamination to the interior contents of the flexible bag 12 but allows for the passage of air or gas. In the embodiments of FIGS. 1A-IE, 2A-2C, 3A-3C, 4A-4C, 9A-9B, 10A, 10B, 11A, 11B, the top of the flexible bag 12 terminates at a seam that includes a hanger sleeve 31 that can accommodate a hanger (e.g., hanging rod). Eyelet(s) 35 may also be provided at the top of the flexible bag 12 to allow for mounting the flexible bag 12 from above (e.g., hanging from a hook).

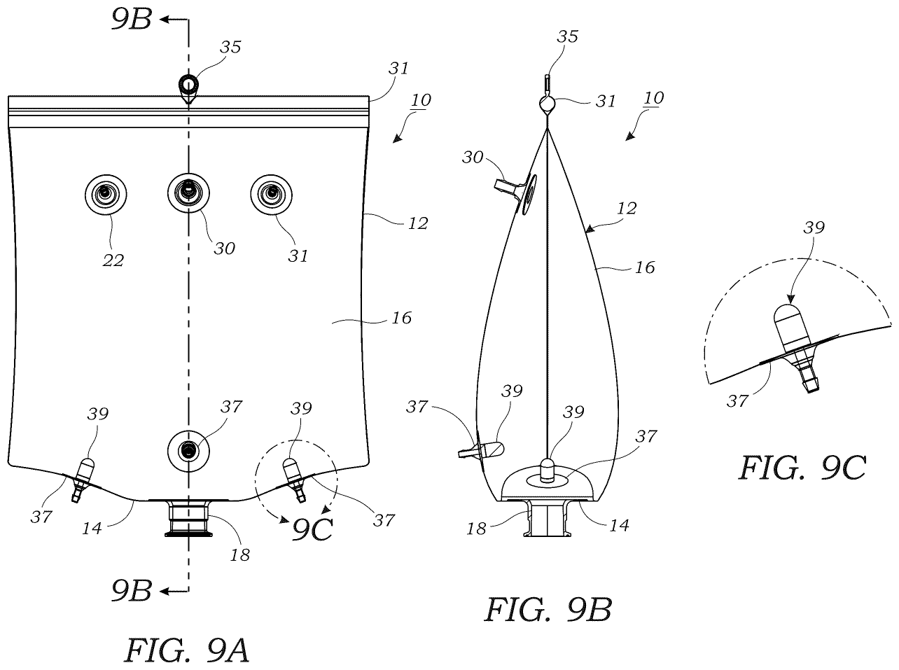

FIGS. 9A-9C illustrate an alternative embodiment of a flexible bag 12 which includes one or more sparger ports 37. Sparger ports 37 allow for the introduction of air or gases into the interior of the flexible bag 12. For example, as seen in FIG. 11A, a source of sparging gas 60 is connected to a sparger port 37 by conduit or tubing 28. The sparger ports 37 may be located on the bottom surface 14 and/or a side surface 16 of the flexible bag 12. The sparger ports 37 include a sparger element 39 that is exposed to the interior of the flexible bag 12 and, in one embodiment, includes one or more holes, apertures, or pores that allow air or gas to bubble into the fluid contained in the flexible bag 12. The sparger element 39 may also include a perforated disc that has holes or pores formed therein. The perforated disc is incorporated into the sparger port 37 and is substantially flush mounted relative to the interior of the flexible bag 12. Conduit or tubing 28 similar to what is used to carry fluid may connect to the sparger port(s) 37 and supply the air or gases. Positive pressure from the air or gases prevents fluid from the interior of the flexible bag 12 from exiting via the sparger port(s) 37.

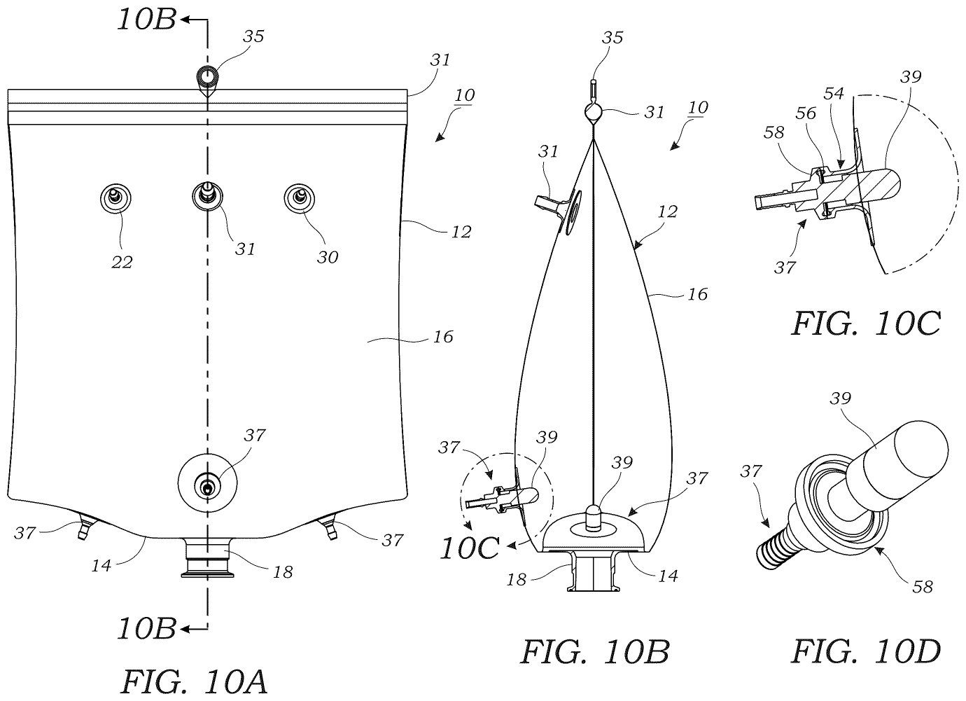

FIGS. 10A-10D illustrate an alternative embodiment of a flexible bag 12 which includes one or more sparger ports 37. In this embodiment, the sparger port 37 includes a tri-clamp port 54 (FIG. 10C) that is secured to the flexible bag 12 and the sparger element 39 is inserted into the sparger port 37 and secured thereto. The tri-clamp port 54 includes a flanged end 56 (FIG. 10D) that engages with an outer flange or lip 58 on the sparger element 39. A sanitary clamp 21 (as illustrated in FIGS. 3A-3C) can be secured around the mating flanged ends to secure the sparger element 39 within the tri-camp port 54. In this particular design, different sparger elements 39 can be inserted to the same tri-clamp port 54. This allows, for example, the use of different sparger elements 39 with different holes, apertures, pores that generate different bubble sizes and/or patterns. Different uses of the flexible bag 12 may require different sparger elements 39.

The flexible bag 12 is typically formed from a polymer material. The polymer material may include a single layer film or multi-layer film. In one example, flexible bag 12 is formed from a multi-layer film layer that includes a durable, ethylene-vinyl alcohol copolymer (EVOH) that is oriented on the outside (non-product contact) of the flexible bag 12 while the inside or product contact side of the flexible bag 12 is made from polyethylene (PE). The return port(s) 22, the inlet port(s) 30, the vent port(s) 33, and/or the sparger port(s) 37 may be made from PE as well. The return port(s) 22, the inlet port(s) 30, the vent port(s) 33, and/or the sparger port(s) 37 may be configured with barbed ends, a nipple, or the like so that the conduit or tubing 28 can be secured thereto. In other embodiments, the flexible bag 12 may be formed from single layer fluoropolymers or multi-layer fluoropolymers. The return port(s) 22, the inlet port(s) 30, the vent port(s) 33, and/or the sparger port(s) 37 may be made from fluoropolymers. The return port(s) 22, the inlet port(s) 30, the vent port(s) 33, and/or the sparger port(s) 37 may be laser welded or otherwise secured to the flexible bag 12 (e.g., using adhesive or the like). The valves 24 located in the return ports 22 or inlet ports 30 may include any number of valve types including, for example, a bleed valve, sample valve, ball valve, plug valve, check-valve, butterfly valve, pinch valve, or diode valve. Diode valves are disclosed in, for example, U.S. Pat. No. 1,329,559, which is incorporated by reference. Other types of diode valves are known including those with nozzle and elliptical configurations. Diode valves preferentially allow fluid to flow in one direction through the valve. The valves 24 may be manually or automatically operated (e.g., through the use of pneumatic or servo-operated valves) or, in some instances, their design (e.g., check-valve or diode valve) provides the valve functionality. For example, check-valves 24 allow fluid to enter the flexible bag 12 while preventing fluid from exiting the flexible bag 12. Such a design allows the conduit or tubing 28 to be removed from the return ports 22 (or inlet ports 30) without fluid exiting the flexible bag 12. The valves 24 are preferably located and/or constructed to have little or no hold-up or dead volume. In one aspect the valves 24 are generally flush-mounted or flush-formed with the sides 16 of the flexible bag 12.

The valves 24 in the return port(s) 22 and/or inlet port(s) 30 may be of the same type or they may be different depending on the return port 22 or inlet port 30. The valves 24, if actuatable, can actuated separately from one another. For example, it may be desirous to turn off valves 24 in the return ports 22 that are located above the fluid level within the flexible bag 12. The valve 24 itself may optionally include a construction that aids in mixing or the creation of a turbulent fluid environment within the flexible bag 12. For example, the ball valve 24 may include multiple fluid pathways or split flows that pass through the ball. The ball valve 24 could be used to adjust the jet or spray response of the ball valve 24 or closed completely. Other valves 24 that spray or direct fluid like a sprinkler within the interior of the flexible bag 12 may be used.

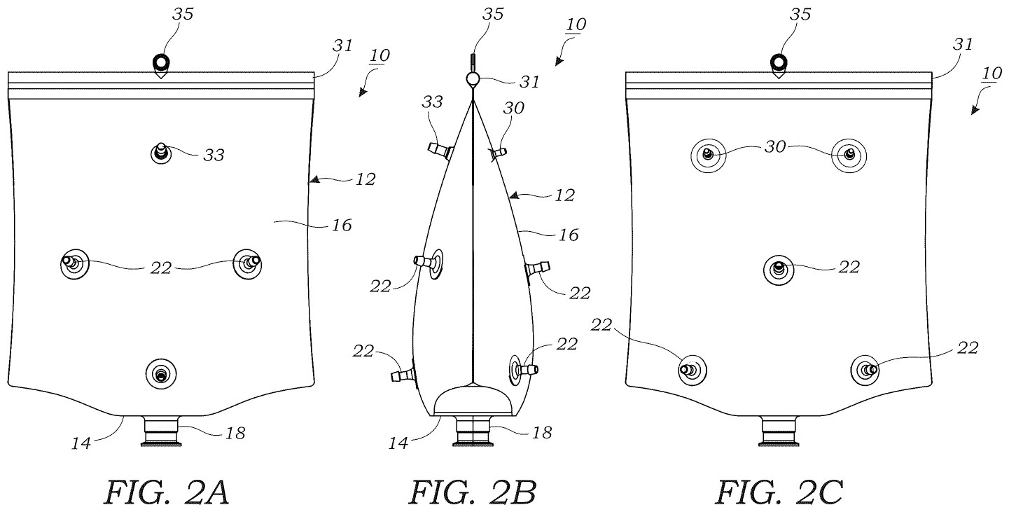

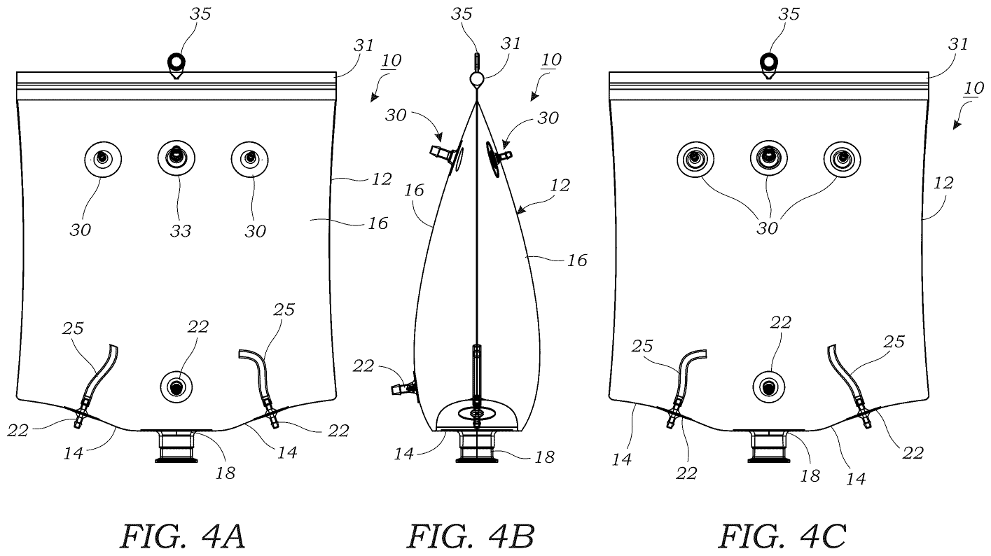

With reference to FIGS. 1A-IE, 2A-2C, and 3A-3C the return ports 22 are located at a lower elevation or height as compared with the inlet ports 30 in one embodiment. In one preferred embodiment, the return ports 22 are located on the side surface(s) 16 adjacent or close to the bottom surface 14. The return ports 22 may also be located in the bottom surface 14 in other embodiments (as seen in FIGS. 4A-4C). The return ports 22 may all be located at different heights or elevations as seen in FIGS. 2A-2C, 3A-3C or, alternatively, the return ports 22 may be located at the same heights FIGS. 1A-1C. In some embodiments, the return ports 22 may be configured to direct the returning flow of fluid into the interior of the flexible bag 12 in a directional manner. For example, the orientation of the return ports 22 (of fluid flow paths therein) may be oriented to create a mixing or turbulent fluid environment within the interior of the flexible fluid container or bag. As an example, fluid entering the interior of the flexible bag 12 may be directed in a clockwise or counter-clockwise arrangement to promote mixing. As another example, multiple fluid jets may be directed at one another or in a specific geometry or orientation to promote mixing.

FIGS. 4A-4C illustrate another embodiment of a single-use flexible container 10 in the form of a flexible bag 12. In this embodiment, a single return port 22 is located in the side surface 16 and two return ports 22 are located in the bottom surface 14 of the flexible bag 12. In this embodiment, the two return ports 22 that are located in the bottom surface 14 include respective segments of tubing 25 through which fluid flows when returning to the flexible bag 12 via the return ports 22. The segments of tubing 25 can be made integral with the return ports 22 or secured thereto (e.g., using a hose barb or other connector). The segments of tubing 25 are flexible and undulate or wave in response to fluid flow. This movement aids in mixing or agitation of the fluid within the flexible bag 12. In other respects, the single-use flexible container 10 is similar to the embodiments of FIGS. 1A-1E and 2A-2C as seen by the similar reference numbers which designate the same components or elements. The segments of tubing 25 may be optional and omitted in other embodiments of the single-use flexible container 10.

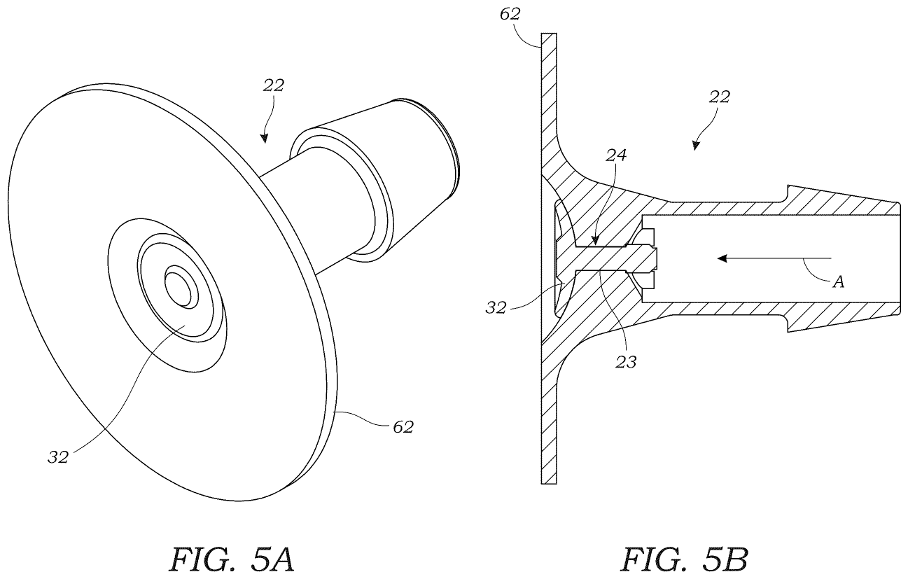

FIGS. 5A and 5B illustrate a return port 22 that includes a valve 24 that functions as a check-valve that allows fluid to flow in one direction (arrow A of FIG. 5A). Fluid can thus enter the flexible bag 12 via the return port 22 but not exit. A rubber or elastic diaphragm 32 is mounted within a flow pathway 23 within the body of the valve 24 (FIG. 5B) and covers the flow pathway 23. Fluid is able to flow in the direction of arrow A and around the rubber or elastic diaphragm 32 but the rubber or elastic diaphragm 32 prevents flow in the opposite direction through the flow pathway 23 in the reverse direction. In particular, the rubber or elastic diaphragm 32 covers the flow pathway 23 and prevents fluid from exiting the return port 22. The return port 22 may include a barbed end or nipple as seen in FIGS. 5A and 5B that can be connected to conduit or tubing 28 such as illustrated in FIG. 3B.

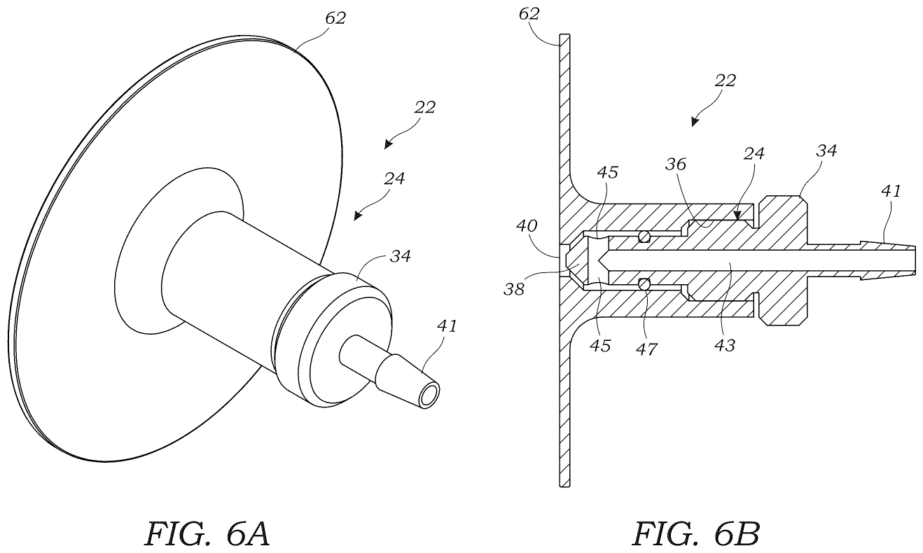

FIGS. 6A and 6B illustrate a return port 22 that includes a valve 24 that is in the form of a sample valve. The valve 24 includes a rotatable stem 34 that engages with a threaded bore 36 and includes an end 38 that forms a seal to prevent the passage of fluid when the rotatable stem 34 is rotated to engage the end 38 with an aperture 40 in the return port 22. The rotatable stem 34 includes a nipple or barbed end 41 that includes a flow pathway 43 inside the rotatable stem 34 that terminates an opening(s) 45 near the end. An O-ring 47 seals the rotatable stem 34 within the bore of the valve 24 as seen in FIG. 6B. To initiate flow, the rotatable stem 34 is rotated to back the end 38 off the aperture 40 so that fluid can flow through the valve 24. That is to say, when the rotatable stem 34 is rotated in the other direction (i.e., in the direction opposite of what closed the valve 24), the end 38 does not form a seal and fluid can then pass through the rotatable stem 34 and through the return port 22 and into the flexible bag 12.

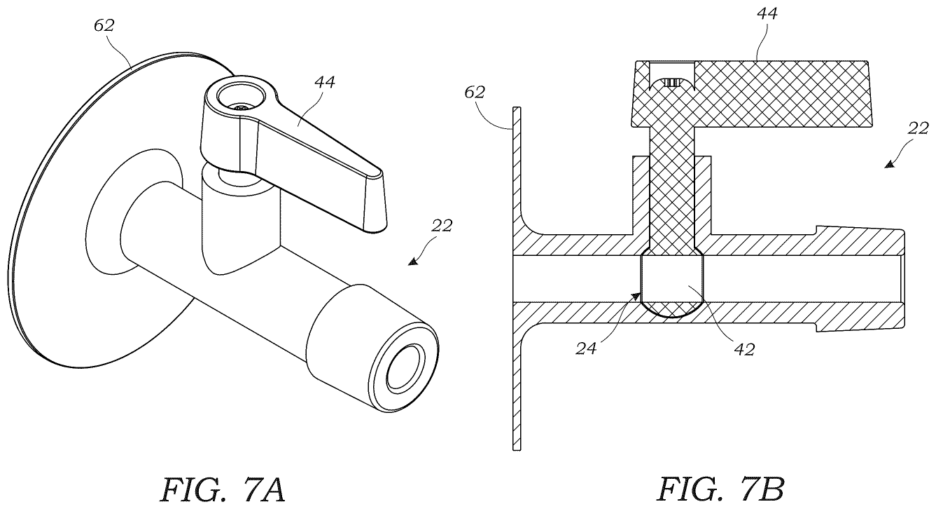

FIGS. 7A and 7B illustrates a return port 22 that includes a valve 24 that is in the form of a ball valve. The valve 24 includes a rotatable ball 42 that includes one or more bores or apertures formed therein to allow passage of fluid when the bores or apertures are aligned with the flow path or lumen of the return port 22. The valve 24 includes a handle 44 that can be manipulated to rotate the ball 42 and turn flow on or off as desired. Of course, the ball valve 24 may also be configured as an automatic ball valve 24 in which case the handle 44 may be omitted. The return port 22 may include a barbed end or nipple as seen in FIGS. 7A and 7B that can be connected to conduit or tubing 28 such as illustrated in FIG. 3B.

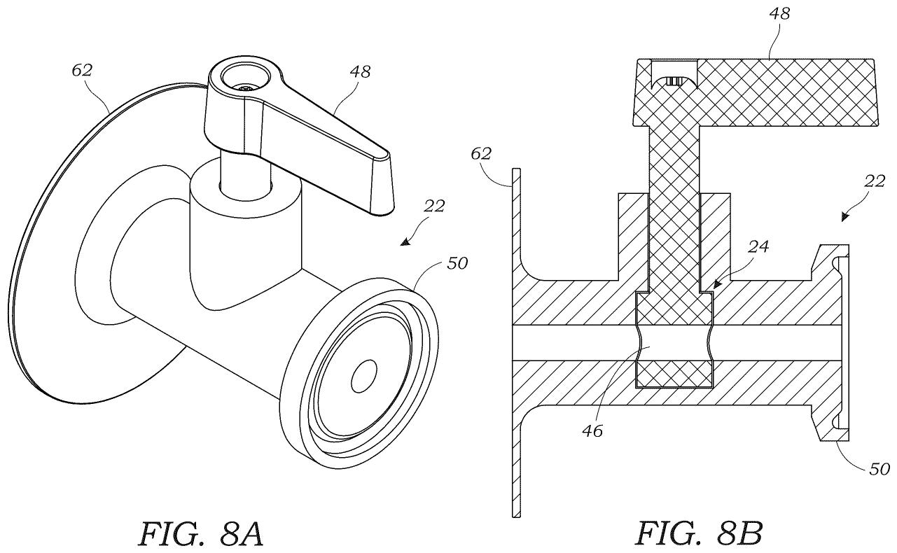

FIGS. 8A and 8B illustrates a return port 22 that includes a valve 24 that is in the form of a plug valve. The valve 24 includes a rotatable plug 46 that includes one or more bores or apertures formed therein to allow passage of fluid when the bores or apertures are aligned with the flow path or lumen of the return port 22. The valve 24 includes a handle 48 that can be manipulated to rotate the plug 46 and turn flow on or off as desired. The handle 48 may be omitted in automatic versions of the plug valve 24. Note that in this embodiment, the return port 22 has a sanitary clamp port fitting 50 instead of the hose barb. The sanitary clamp port fitting 50 may be used with the other return ports 22, inlet ports 30, vent port 33, and/or sparger ports 37 described herein. The sanitary clamp port fitting 50 includes a flanged surface that engages with a corresponding flanged surface of a connecting component. An optional O-ring or seal (not shown) is interposed between the two flanged surfaces to form a fluid-tight seal. A sanitary clamp 21 (like that illustrated in FIGS. 3A-3C) can then be secured over the two coupled flanged surfaces to secure the connection.

In the return ports 22 illustrated in FIGS. 5A, 5B, 6A, 6B, 7A, 7B, 8A, 8B, the return ports 22 may include a flanged end 62. The flange end(s) 62 may be secured to the flexible bag 12 during the manufacture of the flexible bag 12. The flanged end(s) 62 may be welded (e.g., laser weld) or bonded to the flexible bag 12 or, alternatively, the flanged end(s) 62 may be secured to the flexible bag 12 with an adhesive. The flanged end(s) 62 may be secured to an inner surface of the flexible bag 12 or an outer surface of the flexible bag 12. The flanged end(s) 62 allow the return ports 22 to be mounted flush with respect to the flexible bag 12. Similar flanged end(s) 62 may also be used on the inlet ports 30, vent ports 33, and sparger ports 37.

The flexible bag 12 is preferably a single-use or disposable bag that can be discarded after use. The flexible bag 12 may be provided to the end user in an empty state and filled as desired. Alternatively, the flexible bag 12 may, in some embodiments, be prefilled with fluid. In other embodiments, the flexible bag 12 may be empty and then filled with the pump 20 and then optionally removed from the pump 20 with the liquid inside.

To use the single-use flexible container 10, the flexible bag 12 may be secured to the pump 20 using the sanitary clamp 21 (FIGS. 3A-3C). Fluid may be loaded into the flexible bag 12 using one or more inlet ports 30. After filing the flexible bag 12 with fluid, the pump 20 is then used to pump the fluid from inside the flexible bag 12 through the outlets 26 and connected conduit or tubing 28 back into the flexible bag 12 via one or more of the return ports 22. For example, four (4) outlets 26 of the pump 20 are connected to four (4) return ports 22 by four (4) segments of conduit or tubing 28. The pump 20 recirculates fluid through each “arm” of the recirculation flow path. It should be appreciated that in other embodiments one or more of the outlets 26 may not lead back to the flexible bag 12. For instance, in the example above with four (4) outlets 26, one outlet may lead to a downstream or other process while three (3) outlets 26 recirculate fluid back to the flexible bag 12. Additional fluid may be added to the inlet ports 30 or the fluid within the flexible bag 12 may be evacuated over time. Valves 24 may be closed as needed. This may include, for example, when the fluid level falls below the height or elevation where return ports 22 are located, these return ports 22 may be closed. Optionally, air and/or gas may be input into the flexible bag 12 via the one or more sparging ports 37 as explained herein. This could be, for example, air, oxygen, carbon dioxide, or nitrogen that forms bubbles at the sparger element 39. The sparging ports 37 may be coupled to a sparging gas source 60 (FIG. 11A) such as a pressurized tank or the like. After use, the various segments of conduit or tubing 28 may be removed from the respective return port(s) 22, the inlet port(s) 30, the vent port(s) 33, and/or the sparger port(s) 37 and the flexible bag 12 may be detached from the pump 20. The flexible bag 12 can then be discarded.

In one embodiment, and with reference to FIGS. 11A-11B, a separate valve 52 is located in or adjacent to the port or aperture 18. For example, the separate valve 52 may include a valve 52 that is interposed between the inlet of the pump 20 (the pump head of pump 20 is illustrated) and a port 18 that is located in the flexible bag 12. FIGS. 11A-11B illustrate a butterfly valve 52 with a handle 53 (FIG. 11A) that is interposed between the pump 20 and the port 18, although it should be appreciated that other valve types may be used. The separate valve 52 may be secured to the port 18 and/or the pump 20 (e.g., pump head) using a sanitary clamp 21 like that illustrated in FIGS. 3A-3C. This valve 52 enables fluid inside the flexible bag 12 to be isolated from the pump 20. Thus, in one embodiment, the pump 20 may be operated to recirculate fluid inside the flexible bag 12 and, when complete, the separate valve 52 may be closed to prevent fluid from leaving the flexible bag 12 into the pump 20. The valves 24 in the return port(s) 22, the inlet port(s) 30, the vent port(s) 33, and/or the sparger port(s) 37 may be closed and the conduits or tubing 28 removed. The flexible bag 12 with the fluid contained therein can then be removed from the pump 20 and either stored for later use or use in a different process or system. The flexible bag 12 contains the well-mixed fluid that can then be used as needed. In addition, the valve 52 enables an already filled flexible bag 12 to be attached to the pump 20. Here, a flexible bag 12 containing fluid is provided and the port 18 is connected to the pump 20 with the valve 52 in the closed state. Once mounted to the pump 20, the valve 52 can be opened so that fluid can pass from the interior of the flexible bag 12 into the pump 20. FIGS. 11A and 11B illustrate one of the outlets 26 of the pump 20 coupled to conduit or tubing 28 that returns to a return port 22. This allows fluid to be recirculated in the flexible bag 12.

While embodiments of the present invention have been shown and described, various modifications may be made without departing from the scope of the present invention. For example, the types of flexible bags 12 that can be used for the single-use flexible container 10 may vary beyond those specifically recited herein. These include, flexible bags 12 that include a top surface. In addition, valves types other than those specific valves 24 specifically disclosed herein may also be used with the return port(s) 22, the inlet port(s) 30, the vent port(s) 33, and/or the sparger port(s) 37. The invention, therefore, should not be limited, except to the following claims, and their equivalents.

What is claimed is:

1. A single-use flexible fluid container comprising:
a flexible bag having a bottom surface and one or more side surfaces;
a port or aperture formed in the bottom surface of the flexible bag; and
a plurality of return ports disposed in one or more side surfaces or a bottom surface of the flexible bag, each return port comprising a valve therein.

2. The single-use flexible fluid container of claim 1, wherein the plurality of return ports are located at the same elevation or height on the one or more side surfaces.

3. The single-use flexible fluid container of claim 1, wherein the plurality of return ports are located at different elevations or heights on the one or more side surfaces.

4. The single-use flexible fluid container of claim 1, wherein the valve disposed in each return port comprises one of a bleed valve, sample valve, ball valve, plug valve, check-valve, butterfly valve, pinch valve, or diode valve.

5. The single-use flexible fluid container of claim 1, further comprising one or more inlet ports or vent ports disposed in one or more of the side surfaces.

6. The single-use flexible fluid container of claim 5, wherein the one or more inlet ports or vent ports are located at a higher elevation or height than the plurality of return ports.

7. The single-use flexible fluid container of claim 1, further comprising a pump having an inlet and a plurality of outlets, wherein the inlet of the pump is in fluid communication with the port or aperture formed in the bottom surface of the flexible bag and wherein one or more of the plurality of outlets are connected to respective conduits or tubing that connect to one or more of the plurality of return ports.

8. The single-use flexible fluid container of claim 1, further comprising one or more sparger ports disposed in one or more side surfaces or a bottom surface of the flexible bag.

9. The single-use flexible fluid container of claim 1, further comprising a valve disposed in or adjacent to the port or aperture formed in the bottom surface of the flexible bag.

10. A system for mixing fluids comprising:
a flexible bag having a bottom surface and one or more side surfaces, wherein the bottom surface comprises a port or aperture formed therein;
one or more return ports disposed in one or more side surfaces or a bottom surface of the flexible bag, the one or more return ports comprising respective valves therein; and
a pump having an inlet and a plurality of outlets, wherein the inlet of the pump is in fluid communication with the port or aperture formed in the bottom surface and wherein one or more of the plurality of outlets are connected to respective conduits or tubing that connect to the one or more return ports.

11. The system of claim 10, wherein the valve disposed in each return port comprises one of a bleed valve, sample valve, ball valve, plug valve, check-valve, butterfly valve, pinch valve, or diode valve.

12. The system of claim 10, further comprising one or more inlet or vent ports disposed in one or more of the side surfaces.

13. The system of claim 10, wherein a separate valve is disposed in or adjacent to the port or aperture and configured to isolate fluid inside the flexible bag from the pump.

14. The system of claim 10, further comprising one or more sparger ports disposed in one or more side surfaces or a bottom surface of the flexible bag.

15. A method of using a single-use flexible fluid container comprising:
providing a flexible bag having a bottom surface and one or more side surfaces and a port or aperture formed in the bottom surface of the flexible bag, wherein a plurality of return ports are disposed in one or more side surfaces or a bottom surface of the flexible bag, each return port comprising a valve therein;
securing a pump to the flexible bag, wherein the pump comprises an inlet and a plurality of outlets, wherein the inlet is in fluid communication with the port or aperture formed in the bottom surface of the flexible bag;
securing tubing or conduits to one or more of the plurality of outlets and to one or more of the plurality of return ports; and
operating the pump to recirculate fluid into the flexible bag.

16. The method of claim 15, further comprising introducing a sparging gas into one or more sparging ports disposed on the one or more side surfaces or the bottom surface of the flexible bag.

17. The method of claim 15, further comprising removing the flexible bag from the pump.

18. The method of claim 17, wherein a separate valve is disposed in or adjacent to the port or aperture and configured to isolate fluid inside the flexible bag from the pump, wherein the separate valve is closed prior to removing the flexible bag from the pump.

19. The method of claim 18, further comprising securing a second flexible bag filled with fluid to the pump and opening the separate valve.
